(19) United States
(12) Patent Application Publication
(10) Pub. No.: US 2020/0121419 A1
(43) Pub. Date: Apr. 23, 2020

(54) DEVICE HAVING A THERMOCHROMIC TEMPERATURE INDICATOR FOR ACCOMMODATING, HEATING AND APPLYING DENTAL MATERIALS

(71) Applicant: VOCO GmbH, Cuxhaven (DE)

(72) Inventors: Uwe Leiner, Cuxhaven (DE); Manfred Thomas Plaumann, Cuxhaven (DE)

(21) Appl. No.: 16/658,917

(22) Filed: Oct. 21, 2019

(30) Foreign Application Priority Data
Oct. 22, 2018 (DE) 102018126140.3

(51) Int. Cl. A61C 5/62; A61C 5/66; A61K 6/083; A61K 6/09; A61K 6/00

(52) U.S. Cl. CPC A61C 5/62; A61C 5/66; A61C 2202/01; A61K 6/09; A61K 6/0052; A61K 6/083

(57) ABSTRACT

The present invention relates to devices having a thermochromic temperature indicator for accommodating, heating and applying dental materials, to corresponding methods of heating dental materials and of recognizing the temperature of the dental materials, and to a corresponding kit.

### CROSS-REFERENCE TO RELATED APPLICATIONS

This application claims priority to German Patent Application No. 102018126140.3, filed on Oct. 22, 2018, which is herein incorporated by reference in its entirety.

### FIELD OF THE DISCLOSURE

The present invention relates to devices having a thermochromic temperature indicator for accommodating, heating and applying dental materials, to a method of heating dental materials and of recognizing the temperature of the dental materials, and to a corresponding kit.

### BACKGROUND OF THE DISCLOSURE

One example of a device for accommodating and applying a dental material can be considered to be what is called a compule, but other devices, for example syringe-like packings or capsules, are also useful for this purpose.

Compules are sufficiently well known from the prior art as packaging means for dental compositions (EP 1 689 314 A1, EP 2 364 123A1, EP 2 696 794 A1, EP 0 848 935 A1, EP 2 412 448 A2, EP 0 669 113 B1, DE 102 18 859 A1, DE 10 2013 004 077 A1, U.S. Pat. No. 6,877,983 B1). They enable storage of a pre-proportioned amount of filling material for every patient, such that a new, fresh material is used for each individual patient and cross-contamination can be effectively ruled out. They thus enable a hygienic work cycle.

In order to liquefy the dental materials in order to make them more easily expressible and to enable better adaptation behavior in the cavity, different solutions for heating them have been proposed.

U.S. Pat. No. 7,097,452 B2 describes a compule for storing and heating dental composites, the body of which consists of an electrically conductive plastic and a heat-conducting plastic. An external power source is required for heating.

EP 1 740 119 A1 describes a compule for storing and dispensing dental composites, the body of which contains an energy-releasing device. This energy-releasing device is, for example, a heating wire in a spiral arrangement for heating of the compule and its contents.

U.S. Pat. No. 7,086,861 B2 discloses a compule for storing and dispensing dental composites, the body of which includes an electrically operated induction coil that serves to heat the compule and hence its contents.

As well as these specific heatable compules, ovens can also be configured such that they enable the heating of the compules together with their contents.

EP 1 151 728 B1 discloses a method of heating compules and a corresponding heating device.

EP 1 479 356 A2 discloses a further heating device for heating compules together with their contents.

In addition, the as yet unpublished DE 10 2018 114 690 described specially developed dental materials that are notable for a particularly large drop in viscosity when heated. They behave like flow composites at high temperature and adapt very well to the tooth substance in the cavity. At room temperature, they behave like condensable composites, are modelable and have very good mechanical values.

In order to make it possible for the user to see the temperature or viscosity of a heated material from the outside, WO 2010/003190 A1 proposed packings having a thermochromic label.

A disadvantage of these thermochromic labels is that they essentially indicate the temperature of the surface of the packing and not the temperature of the contents.

A further disadvantage is that the compules present on the market are subject to very narrow tolerances with regard to their outside dimensions, from which deviation is possible neither in the upward nor downward direction without resulting in adverse effects on the dispensing devices used for application. The reason for this is that the various dispensing devices attack different parts of the compule body by different accommodating mechanisms and since each—according to the accommodating mechanism—does not work as desired in the event of excessively large dimensions or, in the case of another accommodating mechanism, in the case of excessively small dimensions.

The use of labels in this connection is problematic. Label materials have a thickness in the 1/10 mm range. However, the compules have a wall thickness only in an order of magnitude of about 1 mm. When the compules are used, high material stresses occur, and for that reason they typically have to be manufactured from high-strength plastics. If the intention were thus to use labels, the wall thickness would have to be reduced by the thickness of the label material in order not to increase and hence adversely affect the external dimensions. In the case of a label material of thickness 1/10 mm, the wall thickness must thus be reduced in an order of magnitude of 10%. Thus, the use of thermochromic labels is also disadvantageous for stability of the compule body. A compule body of inadequate stability can burst or break during use. Since this will normally happen within a patient's mouth during application, the result would be an elevated level of risk to the patient.

Since there is a temperature gradient from the outside inward when dental materials are heated in heating ovens, a label on the surface reacts very much more quickly to the rise in temperature than the dental material within the packing. There is therefore the risk that the dentist will take the material out of the heating oven too early and that the material will still not have reached its desired temperature at this time. If the temperature of the material is too low and hence the viscosity of the material is still too high, there is the risk that the material will not adapt optimally in the cavity, with future potential for secondary caries at these sites.

On the other hand, immediately after removal from the heating oven, cooling of the compule occurs. Here too, there is a temperature gradient between the heated contents and the ambient temperature, to which a label on the surface of the packing reacts more quickly than the contents of the packing. The effect of this can be that the label wrongly signals to the dentist that the material has already cooled down again to such an extent that it can no longer be employed even though the material is actually still at the necessary temperature. The result of this is that the material is reheated in the heating oven and the progression of treatment is thus unnecessarily slowed for the patient and the dentist.

There is thus an obvious need for compules that reliably give the dentist information as to whether the contents are ready for use or not.

### SUMMARY OF THE DISCLOSURE

It is an underlying aim of the present invention to provide a device for accommodating, heating and applying a dental material and a method of treating a dental material that avoid the disadvantages of the prior art and give the dentist the necessary certainty for use.

It is therefore desirable to provide a solution that allows simple and practical handling of a dental material for the purposes of accommodation, heating, application and/or treatment thereof.

### BRIEF DESCRIPTION OF THE DRAWINGS

FIG. 1 shows a schematic diagram for illustration of a first working example of the device according to the invention.

FIG. 2 shows a further schematic diagram for illustration of a first working example of the device according to the invention.

FIG. 3 shows a schematic diagram of a device according to the invention in a heating unit.

FIG. 4 shows a further schematic diagram of a device according to the invention in a heating unit.

FIG. 5 shows a further schematic diagram of a device according to the invention in a heating unit.

FIG. 6 shows a further schematic diagram of a device according to the invention in a heating unit.

FIG. 7 shows a temperature profile of dental material, piston and label during a heating and cooling phase.

FIG. 8 shows a schematic diagram of a 2-component injection-molded piston.

FIG. 9 shows a further schematic diagram of a 2-component injection-molded piston.

In the appended drawings and the elucidations relating to these drawings, elements that correspond to one another or are related to one another—where appropriate—are identified by respectively corresponding or similar reference symbols, even if they are to be found in different working examples.

### DETAILED DESCRIPTION OF THE DISCLOSURE

According to the invention, in a first aspect, a device for accommodating and applying dental material as defined in claim 1 is proposed, namely having a cavity for accommodating the dental material and a piston which closes the cavity on one side and which is movable along the longitudinal axis in the device, wherein the piston consists at least partly of a thermochromic material.

The piston preferably has a first color below the switching temperature of the thermochromic material and a second color different than the first color above the switching temperature of the thermochromic material.

The piston preferably has a first color within a first temperature range from 10 to 30° C., preferably within a temperature range from 15 to 25° C., and a second color different than the first color within a second temperature range from 40 to 80° C., preferably within a temperature range from 50 to 70° C.

In a further preferred embodiment, the piston additionally has a third color different than the second color, preferably than the first and second colors, within a third temperature range of greater than 80° C. Such a piston allows the dentist to recognize not just when a dental material is sufficiently hot to be able to work with it, but simultaneously also allows him to recognize whether the dental material is possibly too hot for use on a patient. A reliable indication is thus given to the dentist that he is working within the correct temperature range.

Preferably, the body of the device, over a temperature range from 10 to 80° C., has a color corresponding to the second color of the piston within the second temperature range from 40 to 80° C., preferably within a temperature range from 50 to 70° C.

The piston, along its longitudinal axis, preferably has a transmittance of less than 10%, preferably less than 5%, more preferably less than 1%, for radiation in the wavelength range from 250 to 500 nm, preferably in the wavelength range from 450 to 480 nm.

Preferably, a device according to the invention comprises a water vapor-impermeable and/or opaque packing, meaning that it has been packaged in a water vapor-impermeable and/or opaque manner. Such water vapor-impermeable and/or opaque packings are preferably blister packs or film pouches.

Preferably, a device according to the invention is selected from the group consisting of syringes, application cannulas, capsules and computes.

Even though only thermochromic pigments close to the surface are visible to the user, it has been found that, astonishingly, correlation of the temperature indicated with the actual temperature of the compule contents can be achieved more accurately with a piston that has been fully thermochromically colored than is the case with separate components, for example labels. In addition, the response behavior is by a more direct route in the case of a piston that has been fully thermochromically colored.

It can be assumed that it is the heat transfer within just one material (the piston material) compared to the heat transfer from one material via an adhesive layer to the other material (the label material) that leads to this different response behavior.

In a preferred variant of the above configuration, the piston consists of a plastic colored with a thermochromic material. Preferably, the thermochromic material is selected from the group consisting of inorganic pigments comprising metal salts or metal oxides in which a phase transition, a change in the ligand geometry, a change in the coordination number and/or a change in the crystal field results in a color change, organic pigments comprising thermochromic liquid crystals, conjugated polymers and leuco dyes, and combinations thereof.

Thermochromic dyes used, for the purposes of an example of a thermochromic material that can be utilized in the context of the present invention, may be inorganic or organic pigments.

Inorganic pigments are metal salts or metal oxides in which phase transitions, a change in the ligand geometry, a change in the coordination number or a change in the crystal field results in a color change.

Organic pigments include thermochromic liquid crystals, conjugated polymers and leuco dyes. Thermochromic liquid crystals are, for example, cholesterol derivatives or cyanobiphenyls. Conjugated polymers can have color changes as a result of changes in conformation. Leuco dyes used may be systems based on spiropyrans (formula 1), spirooxazines (formula 2), salicylic Schiff bases (formula 3), bianthrones (formula 4), indolylphthalidenes (formula 5) or fluorans (formula 6).

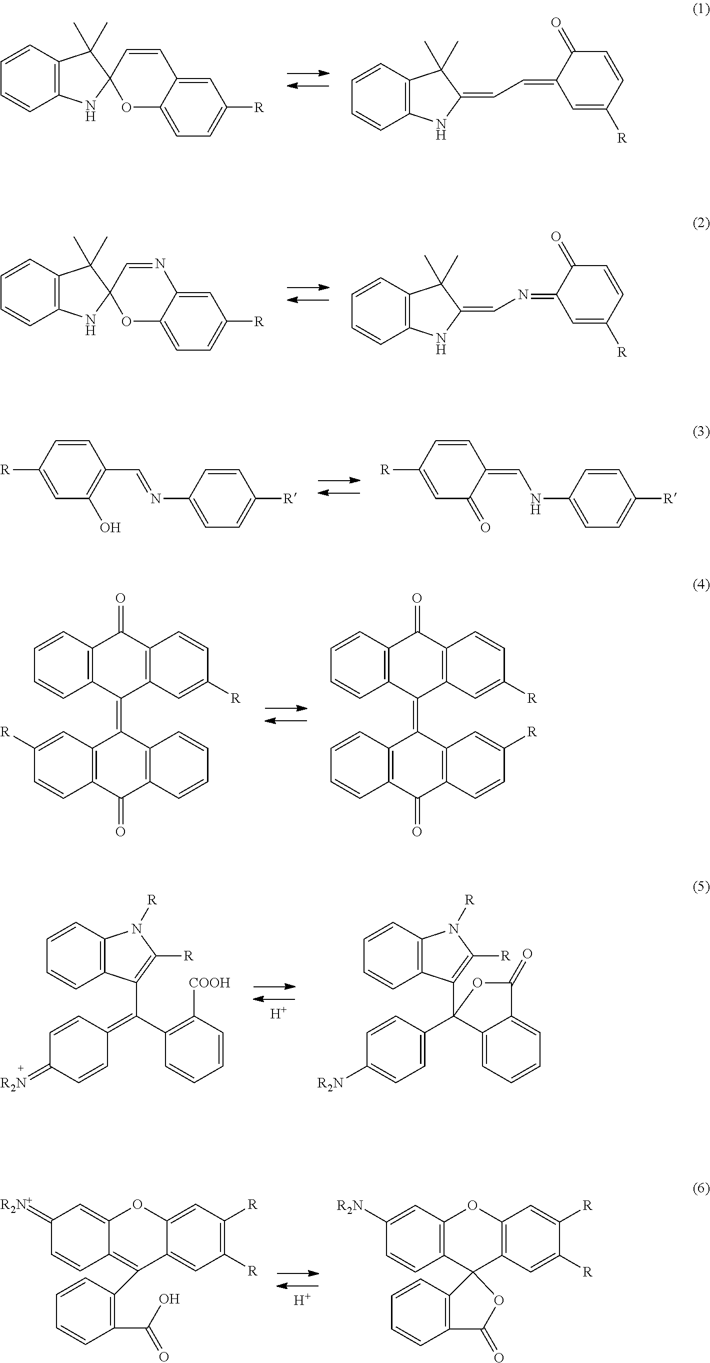

Such systems generally consist of an electron donor (spiropyrans, spirooxazines, salicylic Schiff bases, bianthrones, indolylphthalides or fluorans) and an electron acceptor. In the case of the electron donors, it is possible to establish different colors via suitable choice of substituents. In addition to the positions for the substituents that are shown in simplified form in the formulae, other positions are also conceivable. Electron acceptors used are phenols, azoles or organic acids. Illustrative phenols are phenylphenol, bisphenol A, bisphenol AP, bisphenol AF, bisphenol FL, cresol, resorcinol, phloroglucinol, phenol, phenol oligomers, naphthol, 1,5-dihydroxynaphthalene, pyrocatechol and pyrogallol. Illustrative azoles are benzotriazoles, such as 5-chlorobenzotriazole, 4-laurylaminosulfobenzotriazole, 5-butylbenzotriazole, dibenzotriazole, 2-oxybenzotriazole, 5-ethoxycarbonylbenzotriazole, 5,5′-methylenebisbenzotriazole, imidazoles, such as oxybenzimidazole, and tetrazoles. The organic acids include, for example, aromatic and aliphatic carboxylic acids and substituted derivatives thereof. Examples of aromatic carboxylic acids are salicylic acid, methylenebissalicylic acid, resorcylic acid, gallic acid, benzoic acid, p-hydroxybenzoic acid, pyromellitic acid, naphthoic acid, tannic acid, toluic acid, trimellitic acid, phthalic acid, terephthalic acid and anthranilic acid. Examples of aliphatic carboxylic acids are acids having 1 to 20 carbon atoms, preferably 3 to 15 carbon atoms, for example stearic acid, 1,2-hydroxystearic acid, tartaric acid, citric acid, oxalic acid and lauric acid.

The thermochromic material(s) may be used as such or in microencapsulated form. Advantageously, the thermochromic material or microencapsulated thermochromic material is first used to produce a masterbatch or a colored granulated material, which is then added to the plastic to be colored.

Suitable plastics for the compule body or wall and for the piston of a device according to the invention are the standard thermoplastics polyethylene (PE), polypropylene (PP), polystyrene (PS), polyvinylchloride (PVC) and acrylonitrile-butadiene-styrene copolymer (ABS). Also suitable are the industrial thermoplastics polycarbonate (PC), styrene-acrylonitrile copolymer (SAN), polymethylmethacrylate (PMMA), polyamide (PA), polyoxymethylene (POM), polybutylene terephthalate (PBT), polypropylene terephthalate (PPT), polyethylene terephthalate (PET) and polyphthalamide (PPA). Also suitable are the high-performance thermoplastics polyetherketone (PEK), poly(etheretherketone) (PEEK), poly(etherketoneketone) (PEKK), poly(etheretheretherketone) (PEEEK), poly(etheretherketoneketone) (PEEKK), poly(etherketone-etherketoneketone) (PEKEKK) and polyaryletherketones (PAEK). In addition, it is also possible to use fiber-reinforced plastics.

Preferably, the body or wall of a device according to the invention consists of polyamide (PA) or polybutylene terephthalate (PBT).

Preferably, the piston of a device according to the invention consists of polyethylene (PE), polypropylene (PP), polyvinylchloride (PVC), polystyrene (PS), polytetrafluoroethylene (PTFE), thermoplastic elastomers (TPE), liquid silicone rubber (LSR), silicone, ethylene-propylene-diene rubber (EPDM), nitrile-butadiene rubber (NBR) or fluoro rubber (FKM), preferably of PE, PP or PVC.

It has been found that, when a thermochromic piston is used, there is also the advantage that this can very precisely indicate the temperature of the dental material within the capsule that is in physical contact with the piston. The better the thermal conductivity of the piston material, the more precise and responsive the display.

In a preferred embodiment, the piston may consist of various material combinations. More particularly, the area of the mounted piston directed outward may consist of a layer of a thermochromic material, whereas the rest of the piston consists of a non-thermochromic material that has a better sealing effect, opacity to particular wavelengths or better thermal conductivity than the thermochromic material. The different materials can be cohesively bonded by a 2-component injection molding process, for example, as an indivisible component.

The piston of a device according to the invention is preferably produced by a 2-component injection molding process from two different materials, of which the material facing outward has thermochromic properties.

The material facing inward may also be selected, for example, with improved thermal conductivity properties in order to further improve the response characteristics.

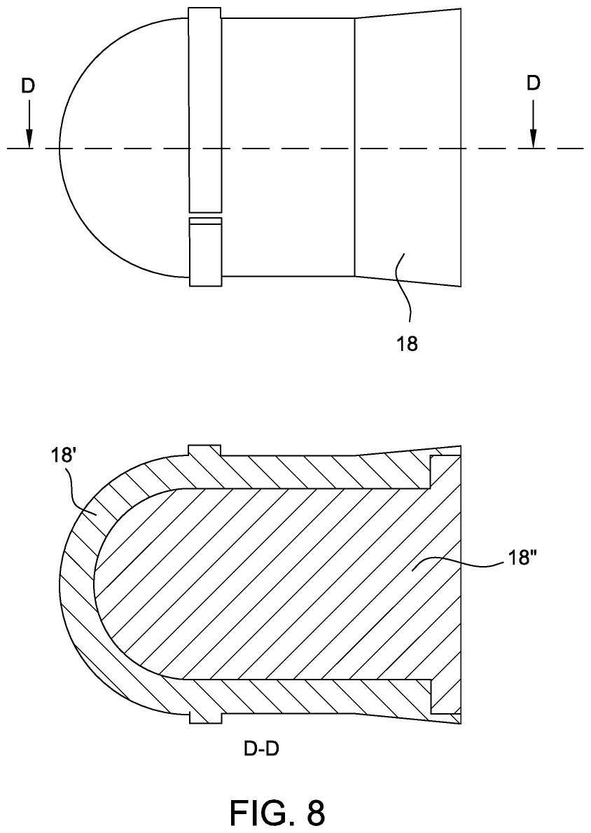

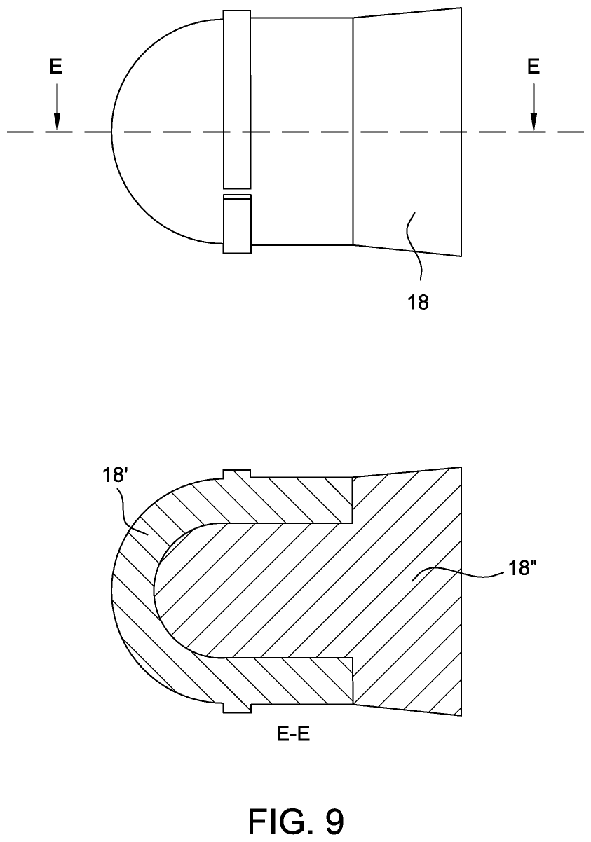

FIGS. 8 and 9 show, in schematic form, such a piston 18 produced by a 2-component injection molding process. One material 18′ faces the interior and the dental material present therein. The second, thermochromic material 18″ faces outward and is visible to the user.

Preferably, a device according to the invention comprises, in the cavity, a radiation-curable dental material which is a one-component composite composition comprising (A) monomers, (B) fillers and (C) initiators.

The dental, light-curable, one-component composite composition, preferably for production of a dental filling material, lining material, luting material or fissure sealant, may in this context comprise, for example:
(A) monomers in an amount of 6% to 35% by weight, based on the composite composition, preferably 10% to 35% by weight, more preferably 10% to 25% by weight,
(B) fillers in an amount of 65% to 93% by weight, preferably 65% to 89% by weight, more preferably 75% to 89% by weight, based on the composite composition,
(C) initiators in an amount of 0.001% to 3% by weight, based on the amount of the composite composition,
(D) further additives in an amount of 0.001% to 5% by weight, based on the amount of the composite composition.

In a particular variant, constituent (A) of the dental, light-curable, one-component composite composition comprises the mixture of at least (A-i) a first monomer substance and (A-ii) a second monomer substance, where the viscosity of the second monomer substance (A-ii) at 20° C. is greater than 100 Pas, the viscosity of the first monomer substance (A-i) at 20° C. is greater than 100 mPa·s, the viscosity of the second monomer substance (A-ii) at 20° C. is greater than that of the first monomer substance (A-i) and the mass ratio of the first monomer substance (A-i) to the second monomer substance (A-ii) is in the range from 2:1 to 1:10, where the second monomer substance (A-ii) preferably contains at least 40% by weight of 2,2-bis[4-(2-hydroxy-3-methacryloyloxypropoxy)-phenyl]propane (bis-GMA) and/or light-curable derivatives of diisocyanatodiphenylmethane (MDI) and/or light-curable derivatives of tetramethyl-m-xylylene diisocyanate (TMXDI), where the percentages by weight are based on the total mass of the monomers (A).

The studies that the inventors conducted in the course of the present invention show that, when constituents (A) to (D) are used in the amounts specified (preferably in the amount ranges specified as preferred), it is possible in a particularly efficient manner to establish envisaged viscosity provisions.

In a preferred configuration, the monomers (A) consist of
(A1) 10% to 60% by weight, preferably 20% to 50% by weight, more preferably 25% to 40% by weight, of light-curable bi- or tricyclic compounds Q(YₓZₑ)_{b} where
   Q denotes a saturated or olefinically unsaturated bi- or tricyclic structural element, each index b is a natural number selected from the group of natural numbers 1, 2 and 3, each Z denotes a light-curable group, each index e is a natural number selected from the group of natural numbers 1, 2 and 3, each Y in the structure Q(YₓZₑ)_{b} with x=1 denotes a structural element that connects the structural element Q to e structural elements Z and denotes a straight or branched alkylene group, where the alkylene group may be interrupted by oxygen atoms and each index x is 0 or 1,
(A2) 40% to 90% by weight, preferably 50% to 80% by weight, more preferably 60% to 75% by weight, of 2,2-bis[4-(2-hydroxy-3-methacryloyloxypropoxy)-phenyl]propane (bis-GMA) and/or light-curable derivatives of diisocyanatodiphenylmethane (MDI) and/or light-curable derivatives of tetramethyl-m-xylylene diisocyanate (TMXDI),
(A3) 0% to 15% by weight, preferably 0% to 10% by weight, more preferably 0% to 5% by weight, of further free-radically polymerizable monomers that cannot be assigned to (A1) or (A2),
   where the percentages by weight of (A1), (A2) and (A3) are based on the total mass of the monomers (A).

The dental, light-curable, one-component composite composition may further comprise, for example, (A) monomers, (B) fillers and (C) initiators, where the monomers (A) consist of
(A1) 10% to 60% by weight, preferably 20% to 50% by weight, more preferably 25% to 40% by weight, of light-curable bi- or tricyclic compounds Q(YₓZₑ)_{b} where Q denotes a saturated or olefinically unsaturated bi- or tricyclic structural element, each index b is a natural number selected from the group of natural numbers 1, 2 and 3, each Z denotes a light-curable group, each index e is a natural number selected from the group of natural numbers 1, 2 and 3, each Y in the structure Q(YₓZₑ)_{b} with x=1 denotes a structural element that connects the structural element Q to e structural elements Z and denotes a straight or branched alkylene group, where the alkylene group may be interrupted by oxygen atoms and each index x is 0 or 1,
(A2) 40% to 90% by weight, preferably 50% to 80% by weight, more preferably 60% to 75% by weight, of 2,2-bis[4-(2-hydroxy-3-methacryloyloxypropoxy)-phenyl]propane (bis-GMA) and/or light-curable derivatives of diisocyanatodiphenylmethane (MDI) and/or light-curable derivatives of tetramethyl-m-xylylene diisocyanate (TMXDI),
(A3) 0% to 15% by weight, preferably 0% to 10% by weight, more preferably 0% to 5% by weight, most preferably 0% by weight, of further free-radically polymerizable monomers that cannot be assigned to (A1) or (A2),
   where the percentages by weight of (A1), (A2) and (A3) are based on the total mass of monomers (A).

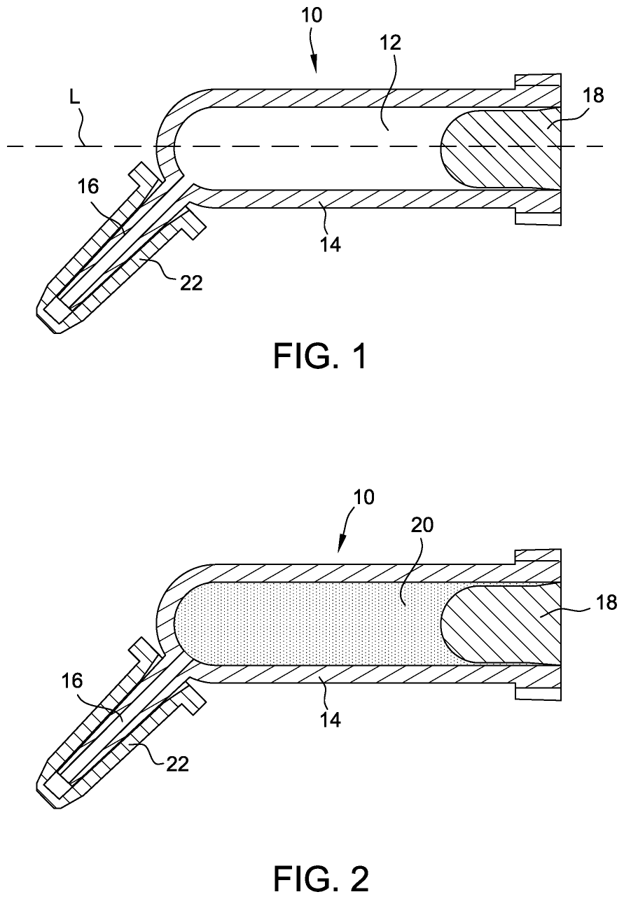

FIG. 1 shows a schematic diagram for illustration of a first working example of the device according to the invention.

In the first working example, the device 10 is designed as a compule having a cavity 12 intended to accommodate dental material (not shown in FIG. 1). The cavity 12 is surrounded by a body 14 of the compule 10, which is in turn provided with an outlet opening 16 closable with a cap 22. The portion of the cavity 12 available is additionally bounded by a piston 18 disposed within the body 14 of the compule 10, in order to be movable along a longitudinal axis L of the compule 10, such that dental material present in the cavity 12 can be dispensed through the outlet opening 16.

Since compules as such and their construction and use are known, no further explanation is required.

However, it should be pointed out that the description of the invention with reference to working examples in the form of a compule should not be regarded as a restriction, since the invention generally envisages a device suitable for accommodating and applying dental material, which can be implemented not just by a compule but also, for example, by means of a syringe, capsule or application cannula.

FIG. 2 shows a further schematic diagram for illustration of a first working example of the device according to the invention and its use. The dental material 20 is shown here in the cavity 12.

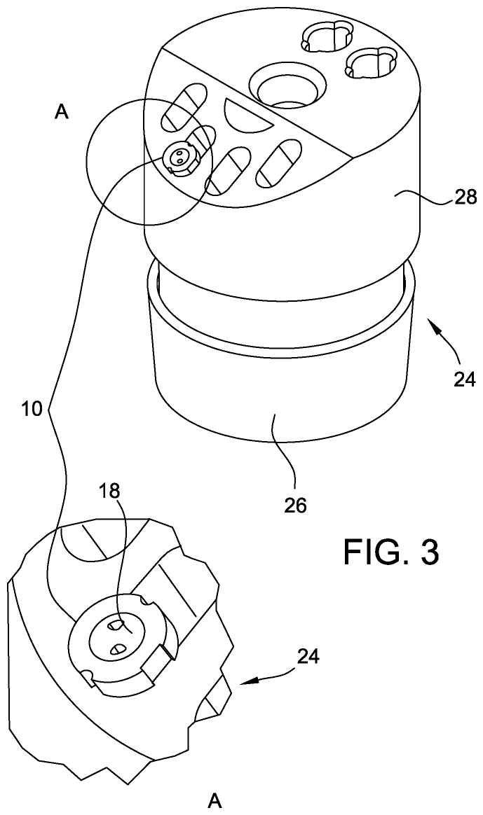

FIG. 3 shows a three dimensional diagram of the inventive device 10 in a heating oven 24. The heating oven 24 consists of a base 26, the heating elements, and measurement and control technology. On the base 26 stands a holding block 28 with various cavities into which a wide variety of different devices according to the invention can be placed.

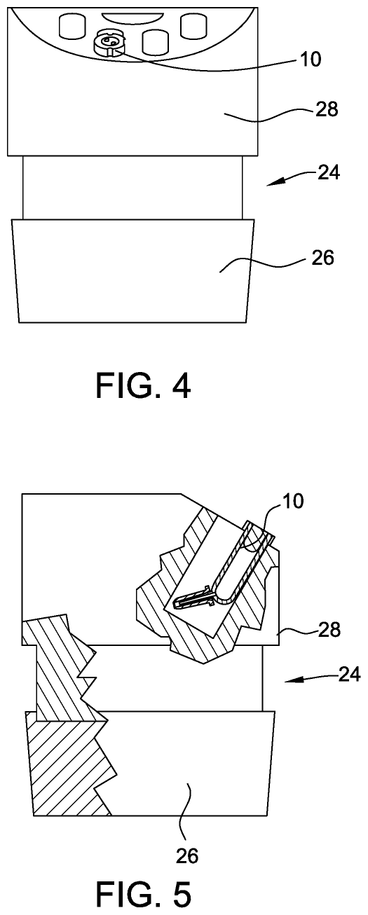

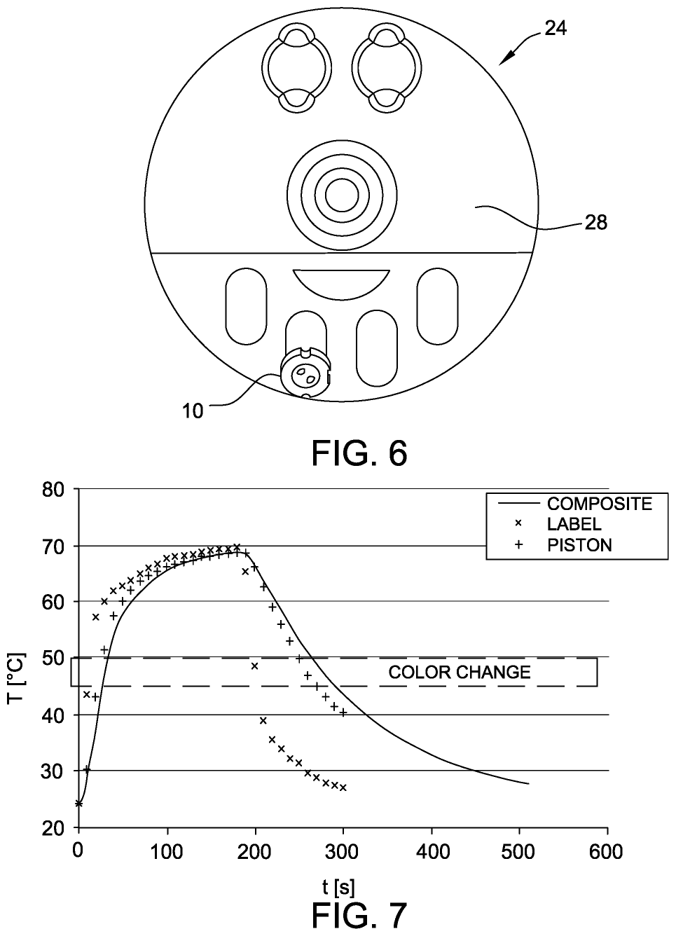

FIGS. 4 to 6 show corresponding diagrams of the inventive device 10 in a heating oven 24 from different perspectives.

In this example, there is a compule 10 with a thermochromic piston 18 in one of the holders. The base 26 is switched on and transmits heat energy to the holding block 28 that stands thereon. The latter is appropriately manufactured from a material of good thermal conductivity, for example a metal. This holding block 28 is thus heated to a preset temperature. As soon as this has been attained, the control technology keeps the temperature constant.

After heating up to 60° C., for example, the user places the inventive device 10 in the holder. The portion of the thermochromic piston (18, 18″) that faces outward is readily visible in this case. The device according to the invention is then heated by the holding block through physical contact, but also via convective and radiative heat. The heat energy penetrates through the wall or the body 14 of the device according to the invention to the dental material 20 present within the interior 12 of the device, but likewise also to the thermochromic piston 18. This heats both the piston and the material. At the changeover point, the piston changes its color, due to its thermochromic properties.

This switching point is chosen such that it indicates a desired minimum processing temperature if the material has a large processing temperature range.

For materials that have a narrow processing temperature range, the switching point is chosen such that it changes over at the desired processing temperature.

For better control, the heating block 24 may contain one or more color markings, with the aid of which the user can compare the complete color changeover with the thermochromic piston. This color marking can also be configured, for example, so as to be exchangeable.

In addition, it is possible to color the compule body such that color of the compule body corresponds to the color of the thermochromic piston that has undergone a change. This eliminates the need to use separate color markings described above.

Preferably, a kit according to the invention comprises

a device according to the invention,

a dental material present in the device and

a heating unit.

Preferably, the heating unit of a kit according to the invention has a reference label, reference marker or reference scale that assigns a temperature to the color of the piston.

Devices according to the invention can additionally be used advantageously in methods of heating dental materials.

Such a method of heating dental materials comprises the following steps:
a) providing a device according to the invention, comprising a dental material,
b) providing a heating unit,
c) placing the device comprising the dental material in the heating unit,
d) heating the device and the dental material present therein from a first temperature below the switching temperature of the thermochromic piston material to a second temperature above the switching temperature of the thermochromic piston material,
e) visually recognizing that a temperature above the switching temperature has been attained by the color change in the thermochromic piston material.

In a preferred method according to the invention, heating is effected in step d) from a first temperature in the range from 10 to 30° C., preferably in the range from 15 to 25° C., to a second temperature in the range from 40 to 80° C., preferably in the range from 50 to 70° C.

In a preferred method according to the invention, in step e), the color of the thermochromic piston material is compared with the color of a reference marker or reference scale, preferably mounted on the heating unit, that relates the color to the piston temperature.

In a preferred method according to the invention, step e) involves visual recognition, using the color of the thermochromic piston material, that the temperature is not higher or lower than the second temperature range from 40 to 80° C.

A preferred method according to the invention comprises the following further steps:
f) dispensing the dental material from the device and
g) curing the dental material by light curing.

Preference is also given to a method of dental treatment having the steps of a method according to the invention, wherein the dental material is introduced into the oral cavity of a patient on or after dispensing from the device, preferably allowed to cool therein, and then cured by light curing.

In a preferred method of dental treatment having the steps of a method according to the invention, the dental material is contacted with a patient's tooth to be treated, preferably as filling material, lining material, luting material or fissure sealant.

### LIST OF REFERENCE NUMERALS

10 compule

12 cavity

14 body

16 outlet opening

18 piston

18′ first piston material

18″ second, thermochromic piston material

20 dental material

22 cap

24 heating unit

26 base

28 holding block

L longitudinal axis

### WORKING EXAMPLES

The working examples were conducted on Grandio SO dental composite (VOCO GmbH). The compule material consists of polyamide (PA). The dental composite is Grandio SO in color A2 (batch 1822301). The heating unit was the Caps Warmer (VOCO GmbH). The heating unit was preheated to 68° C. for 30 minutes.

### Example 1

The piston was removed from a compule of Grandio SO (VOCO GmbH). A thermocouple (DTH-TYPK-4K with NiCrNi type K measurement sensor) was placed in the dental material through this rear opening. The compule was placed in the heating unit preheated to 68° C. (Caps Warmer—VOCO GmbH) and, at the same time, the measurement of temperature was started. After three minutes, the compule was removed from the heating unit and recording of the temperature profile was continued.

### Example 2

5% by weight of thermochromic masterbatch and 95% by weight of polyethylene were used to produce films having a thickness of 100 μm. Labels having a size of 10 mm×10 mm were cut out of these and applied to the side wall of a Grandio SO compule. The compule was placed in the heating unit preheated to 68° C. (Caps Warmer—VOCO GmbH). Every 10 seconds, the temperature of the label was measured (Optris CT LT22 CF). In addition, the color of the label was observed. Within a temperature range from 45 to 50° C., there was a color change from violet to red. After three minutes, the compule was removed from the heating unit and recording of the temperature profile every 10 seconds was continued. Within a temperature range from 45 to 50° C., there was a color change from red back to violet.

### Example 3

5% by weight of thermochromic masterbatch and 95% by weight of polyethylene were used to produce pistons of dimensions corresponding to the pistons of Grandio SO. The piston was removed from a compule of Grandio SO (VOCO GmbH) and replaced by a thermochromic piston. The compule was placed in the heating unit preheated to 68° C. (Caps Warmer—VOCO GmbH). Every 10 seconds, the temperature of the piston was measured. In addition, the color of the piston was observed. Within a temperature range from 45 to 50° C., there was a color change from violet to red. After three minutes, the compule was removed from the heating unit and recording of the temperature profile every 10 seconds was continued. Within a temperature range from 45 to 50° C., there was a color change from red back to violet.

FIG. 7 shows the temperature profile for the dental material/composite material (20), the piston (18) and the label. The label on the surface reacts significantly more quickly to the rise or drop in temperature than the piston (18) and the dental material (20) within the compule, both in the course of heating and in the course of cooling.

1. A device (10) for accommodating and applying dental material (20), comprising a body (14), a cavity (12) for accommodating the dental material (20) and a piston (18) which closes the cavity on one side and which is movable along the longitudinal axis (L) in the device (10), wherein the piston (18) comprises a thermochromic material.

2. The device (10) of claim 1, wherein the piston (18) has a first color below the switching temperature of the thermochromic material and a second color different than the first color, above the switching temperature of the thermochromic material.

3. The device (10) of claim 1, wherein the piston (18) has a first color within a first temperature range from 10 to 30° C. and a second color different than the first color within a second temperature range from 40 to 80° C.

4. The device (10) of claim 3, wherein the piston (18) has a first color within a first temperature range from 10 to 30° C., and a second color different than the first color within a second temperature range from 40 to 80° C. and a third color different than the second color, within a third temperature range of greater than 80° C.

5. The device (10) of claim 3, wherein the body (14), over a temperature range from 10 to 80° C., has a color corresponding to the second color of the piston (18) within the second temperature range from 40 to 80° C.

6. The device (10) of claim 1, wherein the piston (18), along the longitudinal axis (L), has a transmittance of less than 10%, for radiation in the wavelength range from 250 to 500 nm.

7. The device (10) of claim 1, wherein the device (10) comprises a water vapor-impermeable and/or opaque packing.

8. The device (10) of claim 1, wherein the piston (18) consists of a plastic and a thermochromic pigment, wherein the plastic is selected from the group consisting of polyethylene (PE), polypropylene (PP), polyvinylchloride (PVC), polystyrene (PS), polytetrafluoroethylene (PTFE), thermoplastic elastomers (TPE), liquid silicone rubber (LSR), silicone, ethylene-propylene-diene rubber (EPDM), nitrile-butadiene rubber (NBR), and fluoro rubber (FKM).

9. The device (10) of claim 1, wherein the piston (18) is produced by a 2-component injection molding process from two different materials (18′, 18″), of-whieh wherein the material (18″) facing outward has thermochromic properties.

10. The device (10) of claim 1, comprising, in the cavity (12), a radiation-curable dental material (20) which is a one-component composite composition comprising
(A) monomers,
(B) fillers and
(C) initiators.

11. The device (10) of claim 1, wherein the device (10) is selected from the group consisting of syringes, application cannulas, capsules and compules.

12. A method of heating dental materials, comprising the following steps:
a) providing the device (10) of claim 1, wherein the device (10) comprises a dental material (20),
b) providing a heating unit (24),
c) placing the device (10) comprising the dental material (20) in the heating unit (24),
d) heating the device (10) and the dental material (20) present therein from a first temperature below the switching temperature of the thermochromic piston material to a second temperature above the switching temperature of the thermochromic piston material, and
e) visually recognizing that a temperature above the switching temperature has been attained by the color change in the thermochromic piston material.

13. The method of claim 12, wherein heating is effected in step d) from a first temperature in the range from 10 to 30° C. to a second temperature in the range from 40 to 80° C.

14. The method of claim 12, wherein, in step e), the color of the thermochromic piston material is compared with the color of a reference marker or reference scale that relates the color to the piston temperature.

15. The method of claim 12, having the following further steps:
f) dispensing the dental material (20) from the device (10) and
g) curing the dental material (20) by light curing.

16. A method of dental treatment comprising heating a dental material (20) according to the method of claim 12, and introducing the dental material (20) into the oral cavity of a patient on or after dispensing from the device (10) and then curing by light curing.

17. The method of claim 16, wherein the dental material (20) is contacted with a patient's tooth to be treated, as filling material, lining material, luting material or fissure sealant.

18. A kit comprising
a device (10) of claim 1,
a dental material (20) present in the device (10), and
a heating unit (24).

19. The kit of claim 18, wherein the heating unit (24) has a reference label, reference marker or reference scale that assigns a temperature to the color of the piston (18).
